(19) United States
(12) Patent Application Publication
(10) Pub. No.: US 2026/0325313 A1
(43) Pub. Date: Oct. 22, 2026

(54) SKIN-COVERED, LINKAGE-DRIVEN IMPLANTABLE PROSTHESIS

(71) Applicant: THE REGENTS OF THE UNIVERSITY OF CALIFORNIA, Oakland, CA (US)

(72) Inventors: Tyler Clites, Calabasas, CA (US); Gracia Lai, Oakland, CA (US); Kodi Azari, Oakland, CA (US); Lauren Wessel, Oakland, CA (US)

(21) Appl. No.: 19/119,277

(22) PCT Filed: Oct. 10, 2023
(86) PCT No.: PCT/US2023/076502

§ 371 Date: Apr. 8, 2025

Related U.S. Application Data
(60) Provisional application No. 63/378,934, filed on Oct. 10, 2022.

(51) Int. Cl. A61F 2/42; A61F 2/30

(52) U.S. Cl. CPC A61F 2/4241; A61F 2002/30471; A61F 2002/30492; A61F 2002/4253

(57) ABSTRACT

A skin-covered, linkage-driven implantable prosthesis, the prosthesis comprising a first linkage comprising a distal end and a proximal end, wherein the proximal end is configured to fixate into a first proximal bone and comprises a first hinge configured to rotate in parallel with a first proximal joint, a second linkage with a hollow center to configured to allow the first linkage to cross through and comprising a distal end and a proximal end, wherein the proximal end is configured to fixate into a second proximal bone, a second hinge attached to distal end of the first linkage and configured to imitate a second proximal joint, and a tip component configured to imitate a distal phalanx and connected to the first linkage by the second hinge and to the second linkage by a bottom hinge, wherein motion of the prosthesis is allowed through a linkage-driven mechanism.

### CROSS-REFERENCE TO RELATED APPLICATIONS

This application claims priority to U.S. Provisional Patent Application No. 63/378,934 filed Oct. 10, 2022, the contents of which are incorporated by reference herein in its entirety.

### BACKGROUND OF THE INVENTION

Limb amputation typically causes a loss of function that can interfere with activities of daily living. There are approximately two million people in the United States with limb loss, most of them caused by vascular disease and trauma (“Limb Loss Statistics.” Amputee Coalition. https://www.amputee-coalition.org/resources/limb-loss-statistics/#1 (Mar. 1, 2021). Among the traumatic amputations, 78% of the cases involve upper extremities, and 90% of those involve partial hand amputations (Imbinto, Ilario et al. 2016. “Treatment of the Partial Hand Amputation: An Engineering Perspective.” IEEE Reviews in Biomedical Engineering 9:32-48). Although prosthetic devices have greatly advanced in the past decade to replace the function of the amputated limb, hand prosthetic devices remain limited due to the confined space and complexity of the human hand.

The hand is an exceptionally versatile part of the body and has enabled humans to evolve as functional and creative beings. Most everyday activities require the grasp of an object, promoted through the opposition motion of our thumb. Losing the thumb alone causes loss in 40% of the overall hand function (Moran, Steven L., and Richard A. Berger. 2003. “Biomechanics and Hand Trauma: What You Need.” Hand Clinics 19(1): 17-31). Additionally, finger amputation cuts off cutaneous sensations that are provided through a dense and complex sensory receptor system in our fingertips.

Current solutions for thumb amputations can be summarized into passive, body-powered, and electrically powered categories. These solutions have not advanced much over the years in terms of options and functionality. Designing a mechanism that can create diverse and intricate motions is quite challenging due to the confined space in the hand. The most used prosthesis for people with thumb amputations are passive and are attached to the skin by suction or special adhesives. They can be removed easily, which restricts embodiment and connect between the prosthesis and the user. Additionally, they can cause skin irritation and do not provide any sensation to the patient nor restore joint motion. As such, patients with thumb amputations do not typically use a thumb prosthesis on a daily basis.

In addition to prosthetic solutions, there are existing surgical solutions that reconstruct the thumb for people with amputations at the metacarpal joint level. The Gilles cocked hat flip surgery takes the iliac crest bone graft and lengthens the thumb by 2 cm (Manktelow, Ralph T. 1986. “Toe to Thumb Transfer.” Microvascular Reconstruction: 165-83. https://link.springer.com/chapter/10.1007/978-3-642-70329-4_25 (Mar. 26, 2022)). This surgery does not restore joint function and limits the extension of thumb length. Another option is the toe-to-hand transfer procedure (Manktelow 1986). This allows for the restoration of the interphalangeal joint, sensation, and is aesthetically acceptable. However, the donor site morbidity is significant as a toe must be amputated. Current commercial finger prosthetic devices are unable to communicate neural sensation (touch/proprioception) to the human nervous system and cannot integrate with the skeletal system. Without these abilities, the user lacks sensation and embodiment of the prosthesis.

Thus, there is a need in the art for improved prosthetics for thumbs, fingers, and toes. The present invention satisfies that need.

### SUMMARY OF THE INVENTION

Described herein is a skin-covered, linkage-driven implantable prosthesis. The prosthesis comprises a first linkage comprising a distal end and a proximal end, wherein the proximal end is configured to fixate into a first proximal bone and comprises a first hinge configured to rotate in parallel with a first proximal joint. The prosthesis also comprises a second linkage with a hollow center configured to allow the first linkage to cross through and comprising a distal end and a proximal end, wherein the proximal end is configured to fixate into a second proximal bone. A second hinge is then attached to distal end of the first linkage and configured to imitate a second proximal joint. Lastly, a tip component configured to imitate a distal phalanx and connected to the first linkage by the second hinge and to the second linkage by a bottom hinge. The overall motion of the prosthesis is allowed through a linkage-driven mechanism.

Accordingly, also described herein is a prosthetic device. The device includes a first linkage unit having a central body, a distal end and a proximal end, where the proximal end is configured to fixate into a first bone. The device also includes a second linkage unit having a central body, a distal end and a proximal end, where the proximal end is configured to fixate into a second bone different from the first bone. The device also includes a distal component sized and shaped to resemble a distal phalanx. The device includes a first hinge in the first linkage unit connecting the proximal end and the central body, a second hinge connecting the distal end of the first linkage unit and proximal end of the distal component, and a third hinge connecting the distal end of the second linkage unit and the proximal end of the distal component.

In some embodiments, the device has a total extended length of between 3-10 centimeters. In some embodiments, the device has a total diameter of between 5 -25 millimeters. In some embodiments, the device has a length of 10-50 millimeters from the second bone to the distal component. In some embodiments, the second hinge is configured to revolve around the third hinge while simultaneously rotating. In some embodiments, the device is configured to have a larger range of motion when the second hinge is set further forward with respect to the third hinge when at rest. In some embodiments, the second hinge is set slightly to the side of, or straddling, the third hinge. In some embodiments, the first hinge is rotatable up to about 90°. In some embodiments, the second hinge is rotatable up to about 90°. In some embodiments, the device further includes a locking pin. In some embodiments, the device comprises metal articulating surfaces. In some embodiments, at least a portion of the first, second, and third hinges are comprise polyethylene. In some embodiments, the device further includes a coverage protecting each of the first, second, and bottom hinges. In some embodiments, the first bone is a metacarpal bone. In some embodiments, the second bone is a residual proximal phalanx. In some embodiments, the first linkage unit passes through at least a portion of the central body of the second linkage unit.

Also described herein is a surgical method of implanting a skin-covered, linkage-driven implantable prosthesis. The method comprises creating an incision of a radial forearm flap with a proximal end and a distal end. Then, the radial artery is clipped on the proximal end of the radial forearm flap, wherein blood is supplied through the distal end of the flap where the radial artery is left intact. The radial forearm flap is then elevated, and the prosthesis is then implanted on the elevated flap. The method then comprises tubularizing the elevated flap around the prosthesis before the incision is closed. Either in the same or a subsequent procedure, the method then comprises fixating the prosthesis into a medullary canal of a first proximal bone and a second proximal bone, reattaching the skin flap to the residual skin around the bone, and initiating a reinnervation procedure of the flap.

In some embodiments, 6 to 8 weeks may pass between tubularizing the elevated flap and fixating it to the first proximal bone and a second proximal bone to allow for integration with the skin without the interference of motion. In some embodiments, the reinnervation procedure includes anastomosing a lateral antebrachial cutaneous nerve in an “end to end” fashion with a digital nerve. In some embodiments, the reinnervation procedure provides usable sensation to the prosthesis, including differentiating temperature, pressure, and pain. In some embodiments, the prosthesis is fixated into the first proximal bone by at least a screw. In some embodiments, the prosthesis is fixated into the second proximal bone using bone cement.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A depicts an exemplary linkage system and mechanism with fixation points and joints. FIG. 1B shows the motion of the linkage system of Figure IA designed for a prosthesis when the finger is extended. FIG. 1C shows the motion of the linkage system of FIG. 1A designed for a prosthesis when the finger is flexed. FIGS. 1D-1G depict more complex exemplary linkage geometries.

FIG. 2 depicts an exemplary device with a hinge design fixated into first and second bones for a thumb. The extended total length of the implant shown is 63.77 mm, with a 14 mm diameter, and 31.8 mm from the proximal phalanx to thumb tip.

FIG. 3 illustrates the exemplary device of FIG. 2, with about a 60° range of motion of the thumb tip when the metacarpophalangeal joint rotates to about 75°.

FIG. 4 depicts additional views of a thumb implant showing hinge design.

FIG. 5 is another exemplary device with a hinge design fixated into first and second bones for a thumb.

FIG. 6 is a flow diagram exemplifying a surgical method of implanting a skin-covered, linkage-driven prosthesis.

FIG. 7A shows an incision of radial forearm flap. FIG. 7B is an antebrachial cutaneous nerve branch that can be included in the flap. FIG. 7C shows a radial sensory nerve. FIG. 7D illustrates the elevation of the flap. FIG. 7E shows an early prototype of an index finger prosthetic implant on elevated flap, the same method for the thumb prosthesis. FIG. 7F shows the ‘tubularized’ flap around index finger prosthetic device. FIG. 7G shows the linkage driven flexion motion of index finger prosthetic device under the skin flap. FIG. 7H outlines the path of the radial forearm flap to index finger.

FIG. 8A exemplifies the flexor pollicis brevis muscle, the A1 pulley tendon, and palmar digital nerves in a human cadaver. FIG. 8B illustrates an amputated proximal phalanx.

FIGS. 9A and 9B show the implant fixation and insertion points on a cadaver hand.

FIGS. 10A and 10B illustrate the opposition of motion of the implant, wherein the right image shows the implant under the skin.

FIGS. 11A and 11B show the thumb in extension, wherein the right image shows extension of implant under the skin.

FIGS. 12A and 12B show the thumb in flexion, wherein the right image shows flexion of implant under the skin.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of skin-covered, linkage-driven implantable prosthesis. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

Reference throughout the specification to “one embodiment”, “an embodiment” or “some embodiments” means that a particular feature, structure, or characteristic described in connection with an embodiment is included in at least one embodiment of the subject matter disclosed. Thus, the appearance of the phrases “in one embodiment”, “in an embodiment” or “in some embodiments” in various places throughout the specification is not necessarily referring to the same embodiment. Further, the particular features, structures or characteristics of “one embodiment”, “an embodiment” or “some embodiments” may be combined in any suitable manner with each other to form additional embodiments of such combinations. Further, it is intended that embodiments of the disclosed subject matter cover modifications and variations thereof.

As used in the specification and the appended claims, the singular forms “a,” “an,” and “the” include plural referents unless the context expressly dictates otherwise. That is, unless expressly specified otherwise, as used herein the words “a,” “an,”the,“ and the like carry the meaning of ”one or more.“ Additionally, it is to be understood that terms such as ”left,“ ”right.“ ”top,“bottom,”front,“rear,”side,“height,”length,“ ”width,“ ”upper,“lower,” “interior,”exterior,“ ”inner,“outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Linkage Systems and Implantable Prosthetic Devices

Aspects of the invention presented herein include the combination of surgical and mechanical solutions to transfer sensate skin onto an implantable prosthetic device. By utilizing natural human skin, the user (i.e. subject or patient) benefits from innate biological sensory receptors that allow them to feel what the prosthesis is “feeling”. Existing techniques such as prelamination (Chiang, 2006), skin flap transfer (Parrett, Brian M., and Julian J. Pribaz. 2010. “Prefabricated and Prelaminated Flaps.” In Color Atlas of Burn Reconstructive Surgery, Springer Berlin Heidelberg, 300-309. https://link.springer.com/chapter/10.1007/978-3-642-05070-1_34 (Feb. 10, 2021), and reinnervation (Kostakoğlu, N. et al. 1994. “Free Sensate Secondary Skin Flaps: An Experimental Study on Patterns of Reinnervation and Neovascularisation.” British Journal of Plastic Surgery 47(1): 1-9) are integrated into the surgical solution. To replace the function, movement, and shape of the thumb, proposed herein is a new design of an implantable prosthesis that restores joint motion and directly integrates into the bone.

Skin covered prosthetic devices create the illusion of naturally reconstructed sensate limbs. Embodiments of the invention herein focus in part on solutions for people with thumb defects. This includes people with aplasia, hypoplasia, and more commonly thumb amputations.

In some embodiments, the invention is particularly suited for thumb amputations, due to its dramatic impact on function, as well as the relative simplicity of the distal thumb joint. Many activities of daily living require opposition grasp, which is only possible with an independent, actuated thumb; loss of the thumb can reduce overall hand function by as much as 40% (Capek, Karel D., Byron D. Hughes, and Glenn D. Warden. 2018. “Functional Sequelae and Disability Assessment.” Total Burn Care: Fifth Edition: 673-678.el). Amputation of the thumb not only causes challenges in power grip and precision motions, but also limits sensation due to the damage of highly concentrated sensory receptors in the thumb tip. A loss in sensations means a loss in environmental cues, physical connection, and protection from physical damage. Although losing a finger may sound trivial compared to a whole limb loss, our hands are involved in almost all activities of daily living and are always exposed and seen by others. Additionally, the traumatic amputation of the thumb brings about immense psychological impact. Losing a finger is more than a functional loss but also a loss of our self-image and representation to others.

To address the unmet needs of current solutions, proposed herein is an implantable prosthesis that is anchored to bone and covered by native skin. Implanting a prosthetic device beneath the skin allows for direct skeletal attachment, providing mechanical and sensory functionalities from osseointegration without the high risk of infection. Skin replacements may be acquired from locations in the human body where skin is redundant and used as grafts or flaps to replace damaged skin (Doctor, Azmat M., Jimmy Mathew, Sunderraj Ellur, and Anusham A. Ananthram. 2010. “Three-Flap Cover for Total Hand Degloving.” Journal of Plastic, Reconstructive and Aesthetic Surgery 63(4): e402-5. http://dx.doi.org/10.1016/j.bjps.2009.10.007). These skin flaps can be transferred to the amputated site onto the prosthetic thumb. To provide motion and form to the skin, the design of the embodiments of the implantable prosthesis may replace the function of bones, tendons, and shape of the thumb.

As contemplated herein, the invention relates in part to a prosthesis, or prosthetic implant that may be used for any thumb, finger, or toe. In some embodiments, the design may be a thumb prosthesis as described herein. In some embodiments, the prosthetic thumb design may be targeted for people with a functional MCP (metacarpophalangeal joint or the knuckle) joint, where the amputation occurs between the IP (inter-phalangeal) and the metacarpophalangeal joints. Dimensions of the prosthetic thumb include any dimensions suitable for human or other animal use.

In some embodiments, the prosthesis may be skin-covered, linkage-driven and/or implantable. The prosthesis comprises a first linkage comprising a distal end and a proximal end. The proximal end is configured to fixate into a first proximal bone and comprises a first hinge configured to rotate in parallel with a first proximal joint. In some embodiments, the prosthesis also has a second linkage with a hollow center to configured to allow the first linkage to cross through and comprising a distal end and a proximal end, wherein the proximal end is configured to fixate into a second proximal bone. Furthermore, the prosthesis has a second hinge attached to distal end of the first linkage and configured to imitate a second proximal joint. Also, a tip component in the prosthesis is configured to imitate a distal phalanx and connected to the first linkage by the second hinge and to the second linkage by a bottom hinge, wherein motion of the prosthesis is allowed through a linkage-driven mechanism, explained below.

Numerous embodiments of the present invention are designed to restore opposition grasp motion in the hand. This requires a minimal range of motion (ROM), defined as the degree at which the thumb tip rotates. Literature provides the following values for thumb kinematics during opposition: ROM of 44.1°±19.7° for the interphalangeal joint, and 41.6°±12.6° for the metacarpophalangeal joint (Li, Zong Ming, and Jie Tang. 2007. “Coordination of Thumb Joints during Opposition.” Journal of Biomechanics 40(3): 502-10). In addition to a ROM constraint, the design is compact enough to fit under the reconstructed flap. It may allow for osseointegration to the residual proximal phalange, increasing stability and embodiment of the prosthesis. The material of the implant may be biocompatible and provide proper mechanical properties. Ideally, the material may be porous to allow for fibrous ingrowth and stabilization of the implant. However, the joints of the implant may remain non-porous to allow for rotation under the skin.

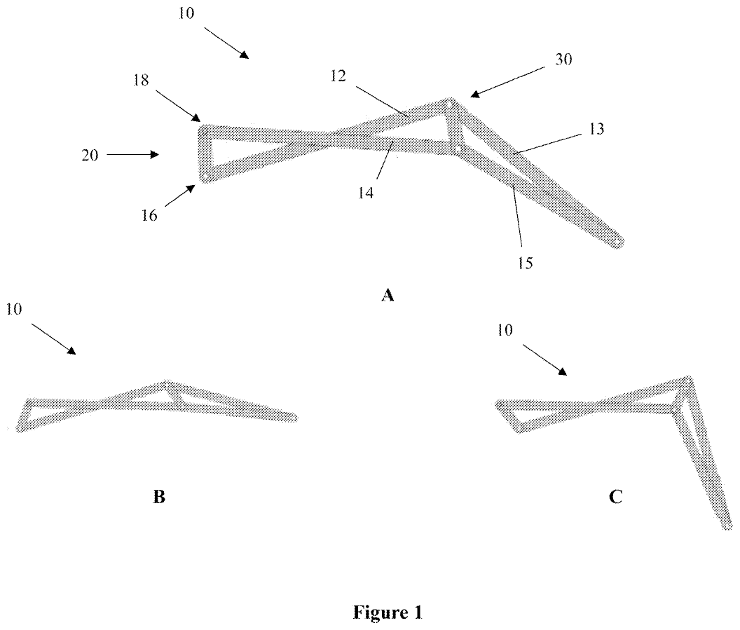

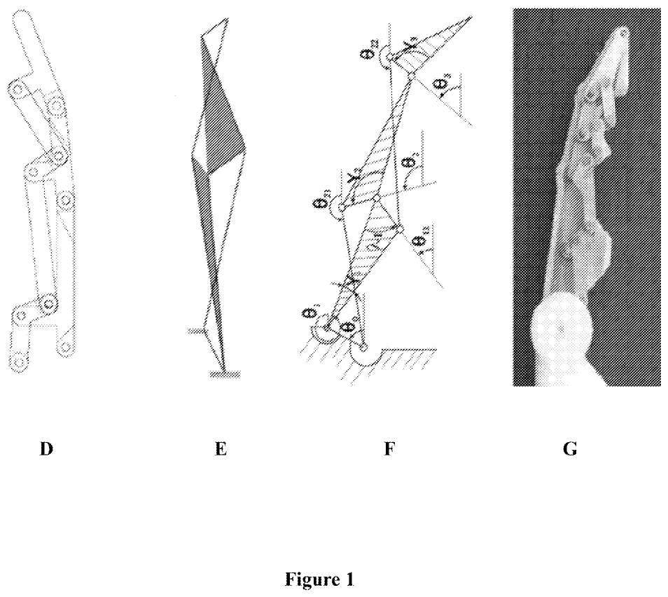

To provide opposition grasp, motion of the inventive device is initiated through a linkage-driven system or mechanism, as shown in FIGS. 1A-1C, In some embodiments, linkage system 10 includes a first linkage component 12 and a second linkage component 14 between a first joint 20 and a second joint 30. In some embodiments, first joint 20 may be the metacarpophalangeal joint (MCP joint) and second joint 30 may be the interphalangeal joint (IP joint). The proximal end 16 of first linkage component 12 and the proximal end 18 of second linkage component 14 at first joint 20 may be ultimately fixed to bone, either directly or with one or more hinging, pivoting, rotating or other secondary components therebetween. In some embodiments, proximal ends 16 and 18 are fixed to different portions of the same bone structure, such as fixation to different locations of the metacarpal, proximal phalanx, or residuals thereof. In some embodiments, proximal ends 16 and 18 are fixed to different bone structures, such as proximal end 16 being ultimately fixed to the metacarpal and proximal end 18 being ultimately fixed to the residual proximal phalanx. It should be appreciated that linkage system 10 may be used to replace any thumb, finger or toe at any anatomical region thereof. Therefore the first and second linkage components can be between any two joints and fixed to the corresponding bone or bones associated with such joints. As contemplated herein, this linkage system 10 as shown in FIG. 1A permits the desired motion shown in FIGS. 1B and 1C, most closely resembling natural movement as compared to existing designs. FIG. 1B shows the motion of the linkage system of FIG. 1A designed for a prosthesis when the finger is extended. FIG. 1C shows the motion of the linkage system of FIG. 1A designed for a prosthesis when the finger is flexed.

In some embodiments, linkage system 10 includes a 4-bar mechanism where the rotation of the metacarpophalangeal joint, or first joint 20, is coupled with the rotation of the interphalangeal joint, or second joint 30. The four bars include first linkage 12, second linkage 14, and bars 13 and 15 distal to the second joint 30. In some embodiments, only a single bar 13 or 15 extends distally from second joint 30. In some embodiments, system 10 may be fixated to the hand in two areas, for example the metacarpal bone (a first proximal bone), and the residual proximal phalanx (a second proximal bone). Due to the fixation sites of the device, rotation of the metacarpophalangeal joint causes first linkage 12 to shift to the right, initiating the linkage-driven mechanism.

It should be appreciated that the linkage system and mechanisms described herein may further include more complex linkage geometries, as shown in FIGS. 1D-IG. These more complex linkage mechanisms may actuate any number of joints in series or in parallel.

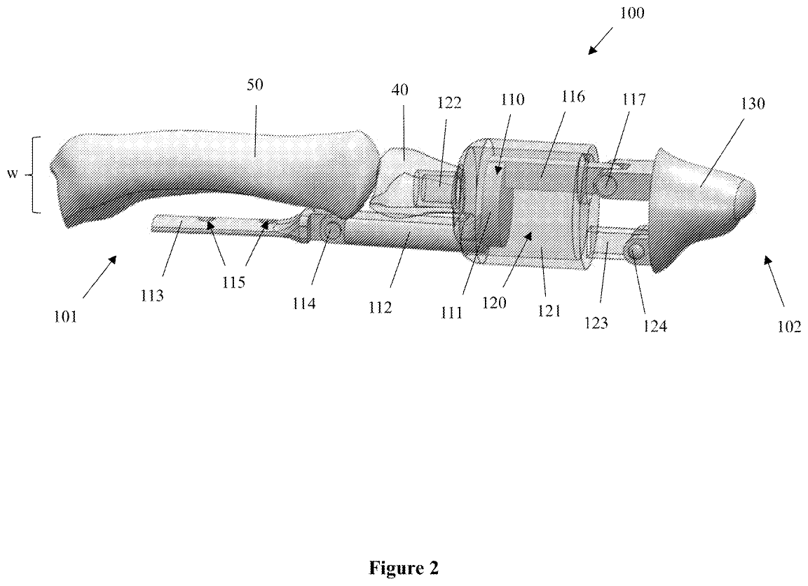

In some embodiments and with reference to FIG. 2, the linkage system is integrated into a device, such as device 100. Device 100 generally includes a first linkage unit 110 and a second linkage unit 120, with an overall proximal end 101 and distal end 102 for reference. In some embodiments, first linkage unit 110 passes through a central housing 121 of second linkage unit 120 (as shown in FIG. 2). In other embodiments, first and second linkage units 110 and 120 are adjacent, or next to each other, such that they are arranged side by side. In some embodiments, first and second linkage units 110 and 120 move independently of each other, or are otherwise not directly connected (mechanically) to each other. In some embodiments, first and second linkage units 110 and 120 have at least one direct mechanical connection or contact point between them.

In some embodiments, first linkage unit 110 includes a central body 111, a proximal arm 112 and a distal arm 116. The overall shape of first linkage unit 110 is not limited, but generally may include a transition from a lower, proximal end to a higher, distal end, the transition ranging generally about the width w of the bone 50 or bones 40 and 50 to which device 100 is fixed or anchored. As such, first linkage 110 includes a proximal anchoring plate 113 with one or more holes 115 through which a screw or other anchoring structure may secure plate 113 to bone 50 and/or 40. In some embodiments, first linkage unit 110 includes a hinge 114 between proximal arm 112 and anchoring plate 113. In some embodiments, there is no hinge, and proximal arm 112 is simply anchored or fixed to bone 50 and/or 40. In some embodiments, anchoring or fixing first linkage unit 110 to bone occurs underneath and along the bone length. In some embodiments, anchoring or fixing first linkage unit 110 to bone occurs at a distal end of the bone. In some embodiments, anchoring or fixing first linkage unit 110 to bone occurs both at the distal end of the bone as well as underneath and along the length of the bone.

In some embodiments, second linkage unit 120 includes a central body 121, a proximal arm 122 and distal arm 123. In some embodiments, central body 121 is a housing with proximal and distal openings through which first linkage unit 110 may pass through. In some embodiments, second linkage unit 120 is generally rod shaped and is positioned adjacent or next to first linkage unit 110. The overall shape of second linkage unit 120 is not limited, but generally may include a transition from a higher, proximal end to a lower, distal end, the transition ranging generally about the width w of the bone 50 or bones 40 and 50 to which device 100 is fixed or anchored. Proximal arm 122 may be anchored to bone, such as bone 40. Anchoring may occur through use of screws or pins, or by insertion into bone and held by friction, adhesive, or the like. In some embodiments, the anchoring point in bone of first linkage unit 110 is different from the anchoring point in bone of second linkage unit 120. In some embodiments, first linkage unit 110 is anchored to a first bone (e.g., bone 50, such as a metacarpal), and second linkage unit 120 is anchored to a second bone (e.g., bone 40, such as a proximal phalanx).

Device 100 may further include a distal component 130, in some embodiments resembling a replacement distal phalanx, connected to the distal ends of both first linkage unit 110 and second linkage unit 120. For example, distal component or phalanx 130 may include a hinge 117 connecting the distal end of first linkage unit 110 to a top region of the proximal end of distal phalanx 130, and may further include a hinge 124 connecting the distal end of second linkage unit 120 to bottom region of the proximal end of distal phalanx 130.

In some embodiments, the extended total length of the device is between 3-10 centimeters. In some embodiments, the device has a total diameter of between 5 -25 millimeters. In some embodiments, the device has a length of 10-50 millimeters from the second bone (e.g. the residual proximal phalanx) to the distal component. In some embodiments, the extended total length of the implant is 63.77 mm, with a 14 mm diameter, and 31.8 mm from the proximal phalanx to thumb tip.

In some embodiments when bone 50 is a metacarpal and bone 40 is a residual proximal phalanx, device 100 includes 3 hinges: an interphalangeal hinge (e.g. hinge 117), a metacarpophalangeal hinge (e.g. hinge 114), and a bottom hinge (e.g. hinge 124) where the interphalangeal hinge represents the interphalangeal joint and the metacarpophalangeal hinge rotates in parallel with the metacarpophalangeal joint. The proximal end of the first linkage unit 110 is screwed into the metacarpal at plate 113 and proximal arm 122 of second linkage unit 120 is fixated into the residual proximal phalanx. Second linkage unit 120 may be designed, in some embodiments, as a hollow cylindrical shape so that at least a portion of first linkage unit 110 can cross through, as shown in FIG. 2. In other embodiments, device 100 may include only 2 hinges.

In some embodiments, the first hinge is rotatable up to about 20°. In some embodiments, the first hinge is rotatable up to about 25°. In some embodiments, the first hinge is rotatable up to about 30°. In some embodiments, the first hinge is rotatable up to about 35°. In some embodiments, the first hinge is rotatable up to about 40°. In some embodiments, the first hinge is rotatable up to about 45°. In some embodiments, the first hinge is rotatable up to about 50°. In some embodiments, the first hinge is rotatable up to about 55°. In some embodiments, the first hinge is rotatable up to about 60°. In some embodiments, the first hinge is rotatable up to about 65°. In some embodiments, the first hinge is rotatable up to about 70°. In some embodiments, the first hinge is rotatable up to about 75°. In some embodiments, the first hinge is rotatable up to about 80°. In some embodiments, the first hinge is rotatable up to about 85°. In some embodiments, the first hinge is rotatable up to about 90°.

In some embodiments, the second hinge is rotatable up to about 20°. In some embodiments, the second hinge is rotatable up to about 25°. In some embodiments, the second hinge is rotatable up to about 30°. In some embodiments, the second hinge is rotatable up to about 35°. In some embodiments, the second hinge is rotatable up to about 40°. In some embodiments, the second hinge is rotatable up to about 45°. In some embodiments, the second hinge is rotatable up to about 50°. In some embodiments, the second hinge is rotatable up to about 55°. In some embodiments, the second hinge is rotatable up to about 60°. In some embodiments, the second hinge is rotatable up to about 65°. In some embodiments, the second hinge is rotatable up to about 70°. In some embodiments, the second hinge is rotatable up to about 75°. In some embodiments, the second hinge is rotatable up to about 80°. In some embodiments, the second hinge is rotatable up to about 85°. In some embodiments, the second hinge is rotatable up to about 90°.

In some embodiments, the third hinge is rotatable up to about 20°. In some embodiments, the third hinge is rotatable up to about 25°. In some embodiments, the third hinge is rotatable up to about 30°. In some embodiments, the third hinge is rotatable up to about 35°. In some embodiments, the third hinge is rotatable up to about 40°. In some embodiments, the third hinge is rotatable up to about 45°. In some embodiments, the third hinge is rotatable up to about 50°. In some embodiments, the third hinge is rotatable up to about 55°. In some embodiments, the third hinge is rotatable up to about 60°. In some embodiments, the third hinge is rotatable up to about 65°. In some embodiments, the third hinge is rotatable up to about 70°. In some embodiments, the third hinge is rotatable up to about 75°. In some embodiments, the third hinge is rotatable up to about 80°. In some embodiments, the third hinge is rotatable up to about 85°. In some embodiments, the third hinge is rotatable up to about 90°.

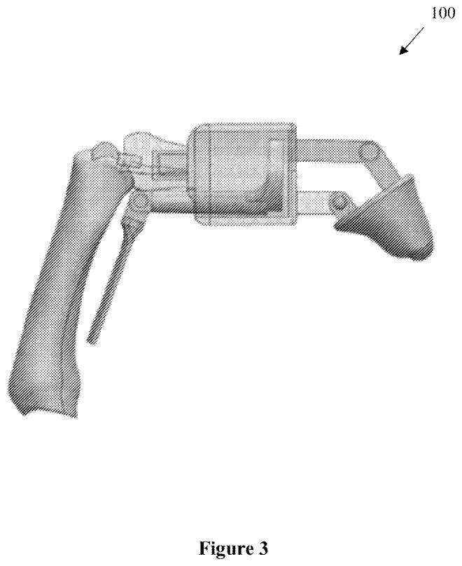

Some embodiments also have flexibility in dimensions and range of motion (ROM). The length and placement of each hinge affects how much the interphalangeal hinge rotates. As the mechanism initiates, the interphalangeal hinge revolves around the bottom hinge while simultaneously rotating itself. The farther forward the interphalangeal hinge is set with respect to the bottom hinge when at rest, the larger the ROM. However, this may also cause the first linkage unit to shift farther down as the metacarpophalangeal joint rotates. In some embodiments, the interphalangeal hinge is set slightly to the left of bottom hinge, allowing the first linkage unit to stay within the bounds of the second linkage unit as the mechanism is initiated. This embodiment allows the metacarpophalangeal joint to rotate up to about 75° before 18 the metacarpophalangeal hinge hits the residual proximal phalange, as shown in FIG. 3. This ROM may be more than sufficient to perform opposition grasp. In some embodiments, as the metacarpophalangeal hinge rotates, the first linkage unit is driven 7 mm forward, allowing the interphalangeal hinge to rotate about 60°.

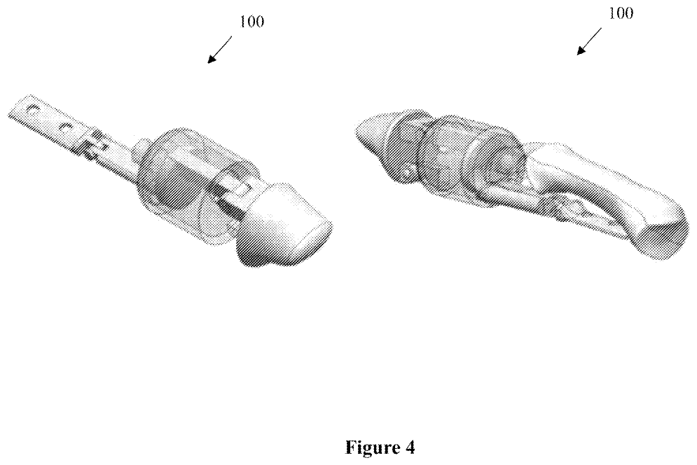

In one embodiment and referring to FIG. 4, the hinge design includes metal (Ti6Al4V) articulation surfaces connected with a locking pin. In another embodiment, part of the hinge may be made of polyethylene, ceramic, metal, or any biocompatible bearing material to create a metal-on-plastic articulating surface, reducing friction and wear.

To shield the hinge mechanism motion from the skin, some embodiments may include a dome-like coverage over the hinges. This reduces chances of extrusion and retain thumb shape at the interphalangeal joint.

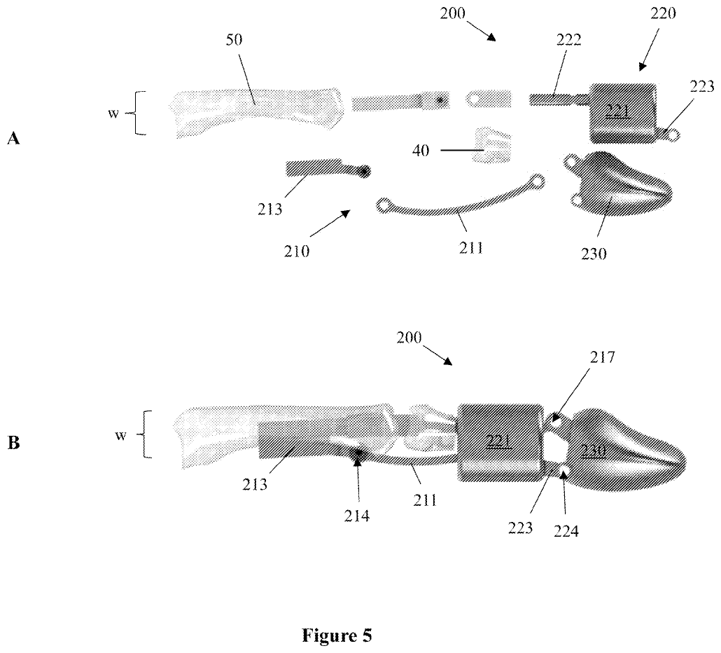

Referring now to FIGS. 5A and 5B is another embodiment, shown as device 200. FIG. 5A is an exploded view of the components of device 200. Unlabeled components were included for purposes of modeling, and in some embodiments they may form part of device 200, while in other embodiments they do not form part of device 200, as they are not required for purposes of functionality and performance. Similar to device 100 of FIG. 2, device 200 includes a first linkage unit 210 and a second linkage unit 220. In some embodiments, first linkage unit 210 passes through a central housing 221 of second linkage unit 220. In other embodiments, first and second linkage units 210 and 220 are adjacent, or next to each other, such that they are arranged side by side. In some embodiments, first and second linkage units 210 and 220 move independently of each other, or are otherwise not directly connected (mechanically) to each other. In some embodiments, first and second linkage units 210 and 220 have at least one direct mechanical connection or contact point between them. In some embodiments, first linkage unit 210 includes a central body or rod 211. The overall shape of first linkage unit 210 is not limited, but generally may include a transition from a lower, proximal end to a higher, distal end, the transition ranging generally about the width w of the bone 50 or bones 40 and 50 to which device 200 is fixed or anchored. As such, first linkage 210 includes a proximal anchoring plate 213 to secure into bone 50 and/or 40. In some embodiments, first linkage unit 210 includes a hinge 214 between rod 211 and anchoring plate 213. In some embodiments, there is no hinge, and rod 211 is simply anchored or fixed to bone 50 and/or 40. In some embodiments, anchoring or fixing first linkage unit 210 to bone occurs underneath and along the bone length. In some embodiments, anchoring or fixing first linkage unit 210 to bone occurs at a distal end of the bone. In some embodiments, anchoring or fixing first linkage unit 210 to bone occurs both at the distal end of the bone as well as underneath and along the length of the bone.

In some embodiments, second linkage unit 220 includes a central body 221, a proximal arm 222 and distal arm 223. In some embodiments, central body 221 is a housing with proximal and distal openings through which first linkage unit 210 may pass through. In some embodiments, second linkage unit 220 is generally rod shaped and is positioned adjacent or next to first linkage unit 210. The overall shape of second linkage unit 220 is not limited, but generally may include a transition from a higher, proximal end to a lower, distal end, the transition ranging generally about the width w of the bone 50 or bones 40 and 50 to which device 200 is fixed or anchored. Proximal arm 222 may be anchored to bone, such as bone 40.

Anchoring may occur through use of screws or pins, or by insertion into bone and held by friction, adhesive, or the like. In some embodiments, the anchoring point in bone of first linkage unit 210 is different from the anchoring point in bone of second linkage unit 220. In some embodiments, first linkage unit 210 is anchored to a first bone (e.g., bone 50, such as a metacarpal), and second linkage unit 220 is anchored to a second bone (e.g., bone 40, such as a proximal phalanx).

Device 200 may further include a distal component 230, for example resembling a replacement distal phalanx, connected to the distal ends of both first linkage unit 210 and second linkage unit 220. For example, distal component 230 may include a hinge 217 connecting the distal end of first linkage unit 210 to a top region of the proximal end of distal phalanx 230, and may further include a hinge 224 connecting the distal end of second linkage unit 220 to bottom region of the proximal end of distal phalanx 230.

Materials for any of the embodiments contemplated herein, including devices 100 and 200, may be selected based on the desired end mechanical properties as well as biocompatibility. Titanium (Ti) and Polyethylene (PE) are common implant materials used for medical and/or dental implants, including artificial joints and bone fixators. The benefit of Ti is that it is highly corrosion resistant and has high biocompatibility. Titanium also allows for osseointegration with the bone (Van Noort 1987). Commercially pure Ti (Ti CP) and extra low interstitial Ti-6AL-4V are two common Ti-based implant biomaterials that are classified as biologically inert. Ti CP, Ti6Al4V, High density polyethylene (HDPE), low density polyethylene (LDPE), and ultra-high molecular weight polyethylene (UHMW), were analyzed for range of motion in Abaqus FEA.

### Methods of Implantation

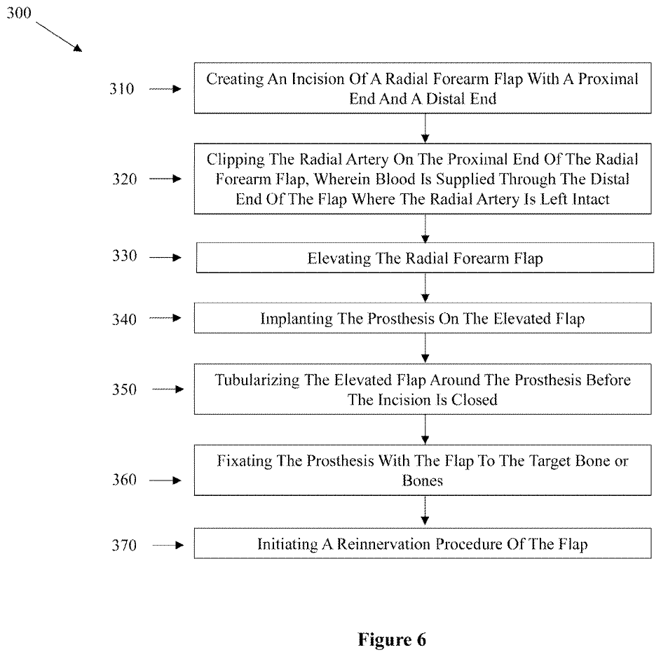

The surgical solution of the human model has been designed and shown in FIG. 6. In some embodiments, a human model procedure may include one or more of the following plastic surgical techniques: prelamination, flap transfer, and reinnervation.

When implanting any foreign material in the body, there may be a concern that the implant can cause extrusion through the skin. Extrusion is when the surface of the implant protrudes the skin and is exposed. Incidences of extrusion are found with mandibular, breast, dental, and ocular implants. Extrusion may be influenced by implant geometry, material, tissue ingrowth, foreign body response, and the amount of trauma in the skin tissue. To minimize the chances of extrusion, the human model presented herein may include a prelamination procedure of the device or prosthesis in the donor site prior to a skin flap transfer. This is where the prosthetic device is implanted under the donor flap for a period of 6 to 8 weeks. The prelamination time allows for the implant to integrate with the skin without the interference of motion, which minimizes disruption of soft tissue ingrowth. In addition, vascularization of the flap may be stabilized which reduces morbidity of the donor site (Fermi, Matteo et al. 2021. “Prelaminated Flaps in Head and Neck Cancer Reconstructive Surgery: A Systematic Review.” Microsurgery 41(6): 584./pmc/articles/PMC8518088/ (Mar. 26, 2022)).

Furthermore and in some embodiments, the radial forearm flap (RFF) may the most viable and appropriate choice for a skin flap transfer as it may be used as a reversed radial forearm rotation flap. This means that the radial artery may be clipped on the proximal end of the flap and blood may be supplied through the distal end of the flap where the radial artery is left intact. After the flap is elevated, the prosthetic may then be implanted. The flap is then ‘tubularized’around the thumb, finger, or joint before the incision is closed.

After 6 to 8 weeks, in some embodiments, a secondary surgical procedure may be conducted to rotate the ‘tubularized’ flap and implant it onto the residual thumb, finger, or joint. In some embodiments, the prosthesis may be fixated into the medullary canal of the residual proximal phalanx and the metacarpal bone.

Accordingly and with reference to FIG. 6, included herein is a surgical method of implanting a skin-covered, linkage-driven implantable prosthesis 300. At step 310, an incision is created of a radial forearm flap with a proximal end and a distal end. At step 320, the radial artery is clipped on the proximal end of the radial forearm flap, where blood is supplied through the distal end of the flap where the radial artery is left intact. At step 330, the radial forearm flap is elevated. At step 340, the prosthesis is implanted on the elevated flap. At step 350, the elevated flap is tubularized around the prosthesis before the incision is closed. At step 360, the prosthesis is fixed or anchored to the target bone or bones. In an example, the prosthesis is a thumb prosthesis, and the prosthesis is fixed with the flap into a medullary canal of a residual proximal phalanx and the metacarpal. At step 370, a reinnervation procedure of the flap is initiated and performed.

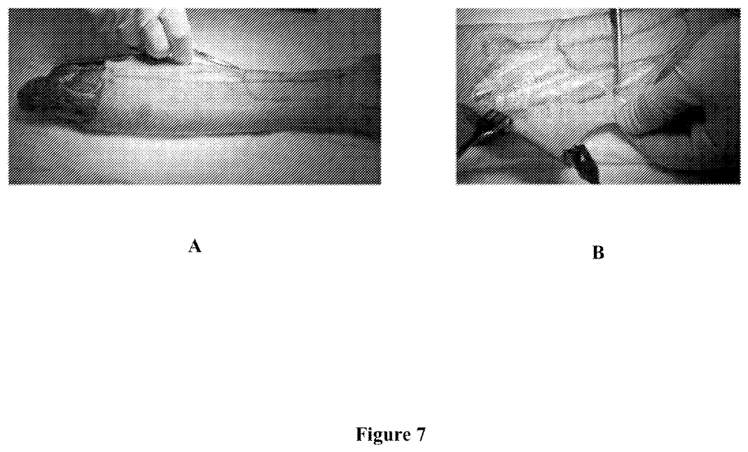

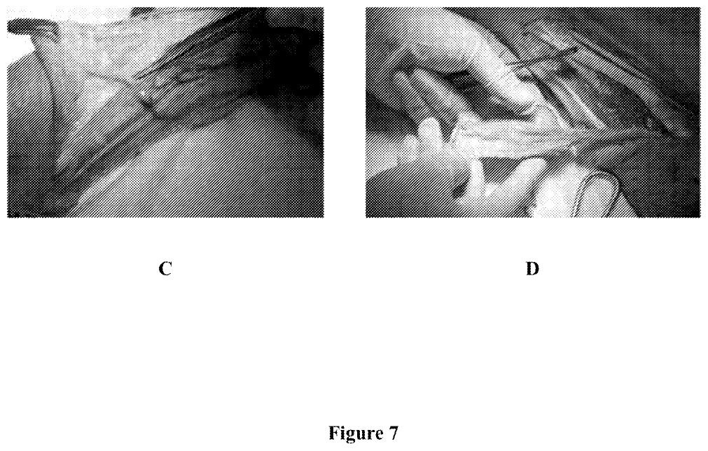

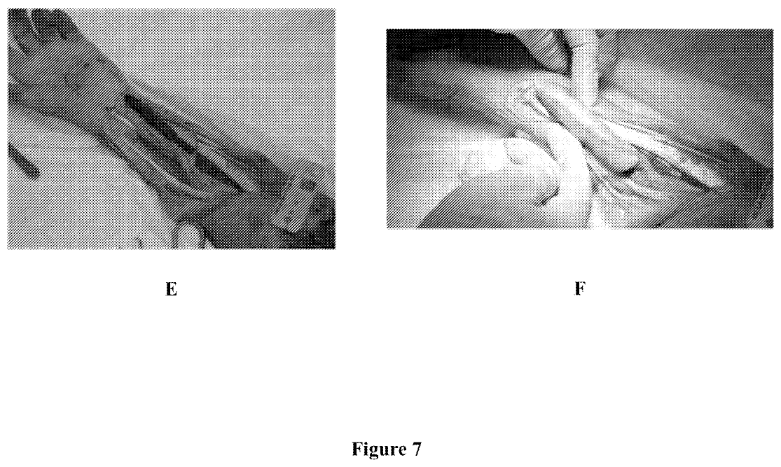

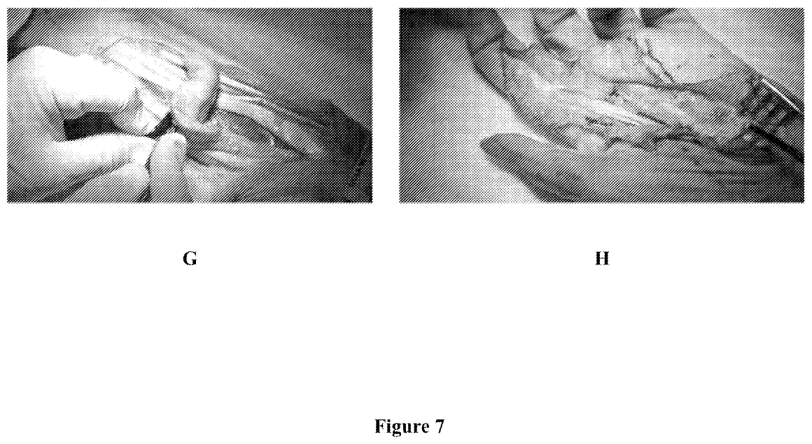

Shown in FIGS. 7A-H are the human cadaver dissection images for an index finger prosthetic device implantation procedure as described above. The procedure for prosthesis implantation described herein is similar to the procedure shown.

The difference between the procedure for thumb versus the one for index finger shown in FIGS. 7A-H is that the radial forearm flap may not be used as a rotation flap for the index finger. Instead, it may be used as a free flap where the blood supply is clipped, and the flap is completely isolated before transferring to the residual index finger. The blood supply may have to be anastomosed at the recipient site.

In some embodiments, to provide native sensation in the flap, reinnervation procedure of the RFF flap may occur when the flap is transferred. This is where the lateral antebrachial cutaneous nerve (LACN) may be anastomosed in an “end to end” fashion with the digital nerve. Other nerves may be used but, in this embodiment, a lateral antebrachial cutaneous nerve is used. Reinnervation may provide usable sensation in which the patient is able to differentiate temperature, pressure, and pain for protection in their reconstructed thumb. To optimize the sensation in the finger, a FDMA flap (First Dorsal Metacarpal Artery Island Flap) or Littler flap transfer procedure may occur subsequently to the secondary procedure. This procedure is often used in thumb injuries, where the dorsal skin of the proximal phalanx of either the index or middle finger is transferred onto the thumb tip (Prabhu, Mahesh, Rajesh Powar, and Sanjitsingh R Sulhyan. 2013. “FDMA FLAP: A VERSATILE TECHNIQUE TO RECONSTRUCT THE THUMB.” Int. J. Pharm. Med. & Bio. Sc. www.ijpmbs.com (Mar. 26, 2022)).

In cases with severe skin loss where tissue engineering solutions are inadequate, reconstructive plastic surgery techniques may be used for skin replantation. A skin flap is a piece of skin removed from one part of the body to replace or repair damaged skin in another area. Degloving of the hand is an example of an injury that requires skin flap transfer. This is a severe injury where the top layers of the skin are separated from the muscle, connective tissue, and bone. Restoring skin on a partial degloved hand may be relatively straightforward, however, to restore skin on an entire degloved hand may require multiple donor skin flaps, adding complexity to the surgery. For an injury where degloving of the entire hand occurs, three skin flaps may be required to reconstruct the hand (Doctor, Azmat M., Jimmy Mathew, Sunderraj Ellur, and Anusham A. Ananthram. 2010. “Three-Flap Cover for Total Hand Degloving.” Journal of Plastic, Reconstructive and Aesthetic Surgery 63(4): e402-5.http://dx.doi.org/10.1016/j.bjps.2009.10.007). The goals of reconstruction in degloving cases can be summarized into 4 areas: prevent loss of function/restore maximum function, provide a stable hand with sensation, prevent stiffness, and achieve good pinch and grasp motions.

There may be several flap options for hand re-surfacing used in plastic surgery. These include free flaps, which are tissue without vascular attachment. Examples include anterolateral thigh flap, radial forearm flap, lateral arm flap, scapular flap, etc. (Doctor et al. 2010). Skin flaps may be bulky, which may restrict movement and decrease the aesthetic of the hand. Flap defatting or thinning may be done where parts of the flap are excised. The radial forearm is a popular area to obtain the flap as it is very thin, pliable, and can be used to cover the thumb, finger, or joint and first webspace.

A degloving case occurring distal to the wrist of the dominant right hand, showed successful reconstruction results, in which the patient was able to lift objects and write legibly (Doctor et al. 2010). The flaps used herein may include the radial forearm flap that covered the first web space and thumb, finger, or joint and the groin-hypogastric flap that covered the remnant fingers.

Flap prefabrication and prelamination may be two methods that are used for skin reconstructive needs. Flap prefabrication is when an axial blood supply is implanted into the donor tissue and is allowed to mature and neovascularize before being transferred to the recipient site. Flap prelamination is characterized by the implantation of tissues or devices in a vascular area prior to flap transfer (Pribaz, Julian J., Neil Fine, and Dennis P. Orgill. 1999. “Flap Prefabrication in the Head and Neck: A 10-Year Experience.” Plastic and Reconstructive Surgery 103(3): 808-20. https://pubmed.ncbi.nlm.nih.gov/10077069/(Feb. 10, 2021)).

To begin the prefabrication process, the requirements of reconstruction and available donor sites may first be defined. The color and texture of the donor site should match with the recipient site. This prefabrication process takes around 8 weeks and involves two stages. The first stage is where a vascular pedicle, which may include the artery and venae surrounded by adventitial tissue is dissected out and transferred to the desired area of tissue. The surrounding tissue of the implanted pedicle may spontaneously connect to the pedicle. Tissue expansion starts around week 1, where a tissue expander is implanted under the desired flap. The second stage is the transfer of flap after 8 weeks of maturation. A conservative 2:1 ratio (length of flap vs. extent of pedicle length within flap) may be used to estimate the size of the flap in clinical cases. (Parrett and Pribaz 2010).

The outcome of prefabrication process reported in literature have been positive and mostly involve reconstruction of the face and neck areas for burn victims. Examples include clinical cases of two patients suffering from extensive burns that underwent prefabrication process in the face and neck area. Results of these clinical cases showed successful transfer and healing of the skin (Parrett and Pribaz 2010).

The prelamination process also involves two stages. The first stage is where the graft material is introduced into the flap. A tissue expander may be used if needed. The location of the radial artery is a common territory used for prelamination. The second stage is flap transfer which occurs after a period of 3-4 weeks. (Parrett and Pribaz 2010).

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

A study was conducted to compare prefabrication and prelamination on 32 patients needing intraoral reconstruction after radical oncological surgery. Results showed that prelaminated fasciomucosal flaps experienced a higher shrinkage rate and early wound healing difficulties in comparison to fasciocutaneous non-prelaminated flaps. Donor site problems occur more frequently in non-prelaminated cases (Poeschl, P. W. et al. 2003. “The Radial Free Forearm Flap-Prelaminated versus Non-Prelaminated: A Comparison of Two Methods.” International Journal of Oral and Maxillofacial Surgery 32(2): 159-66). In order to reduce donor site morbidity, epifascial dissection (Webster, H. R., and D. W. Robinson. 1995. “The Radial Forearm Flap without Fascia and Other Refinements.” European Journal of Plastic Surgery 18(1): 11-13) or de-epithelialization methods may be used to harvest the flap (Kawashima, Takao et al. 1989. “Intraoral and Oropharyngeal Reconstruction Using a De-Epithelialized Forearm Flap.” Head & Neck 11(4): 358-63. http://doi.wiley.com/10.1002/hed.2880110413 (Feb. 12, 2021)).

In an embodiment, by fixating the thumb implant into the residual proximal phalanx, the user benefits from embodiment and stability of the prosthesis through osseointegration. Osseointegration is defined as the direct attachment of an implant to a bony residuum by the formation of bony tissue around the implant without the growth of fibrous tissue at the bone-implant interface (Manurangsee, P., C. Isariyawut, V. Chatuthong, and S. Mekraksawanit. 2000. “Osseointegrated Finger Prosthesis: An Alternative Method for Finger Reconstruction.” Journal of Hand Surgery 25(1): 86-92).

This method may be used for dental protheses as well as bone-conducting hearing aids. Osseointegration used on a finger prothesis may involve the insertion of a threaded titanium implant in the medullar canal of the bone structure and locking the prothesis in place using a small transverse screw device (Jan de Cubber, Zaventum. 2007. “Patent Application Publication (10) Pub. No.: US 2007/0213831 A1.” Us 2007/0213831 a1 1(60): 19-21). For the patient to have adequate function of the prosthesis, they must have a movable metacarpophalangeal (MCP) joint.

Reinnervation may help retore sensation in a skin flap, which may be achieved through connection of nerves in the skin flap with nerves at the recipient site. This may be a useful technique that can be used in our solution to optimize the sensation in the flap after it has been transferred onto the prosthetic thumb, finger, or joint.

Reinnervation may be effective because the neurons in the peripheral nervous system, unlike the central nervous system, can regenerate due to the fast cleanup of damaged cells and assistance of Schwaan cells. Regeneration may occur at a rate of 1 mm/day as long as the soma is not damaged. (“Peripheral Neurological Recovery and Regeneration|PM&R KnowledgeNow.” https://now.aapmr.org/peripheral-neurological-recovery-and-regeneration/ (Jun. 2, 2022).) When an injury that separates the proximal and distal ends of the nerve occurs, the distal end of the nerve undergoes Wallerian degeneration, in which the distal end of the nerve dies. After the degeneration of the distal nerve ending, neuronal regeneration occurs at the proximal end of the injury.

Neurotmesis is the most severe injury of the nerve and occurs during amputations. The return of functionality depends on the severity of neurotmesis: Endoneurium→fair growth, Perineurium→poor growth, and Epineurium→no growth.

There may be 2 different modes of cutaneous sensory reinnervation: the regenerative growth of injured nerve, and the collateral sprouting of neighboring intact nerves. In traumatic or surgical nerve injuries of the hand, partial recovery of sensations are observed long before nerve regeneration can occur (Inbal, Rivka et al. 1987. “Collateral Sprouting in Skin and Sensory Recovery after Nerve Injury in Man.” 28:141-54). This finding contributed to the conclusion that recovery of sensory functions is due to collateral sprouting, in which innervated and denervated tissues adjoin, in the absence of nerve regeneration (Rajan, Bindu et al. 2003. “Epidermal Reinnervation after Intracutaneous Axotomy in Man.” Journal of Comparative Neurology 457(1): 24-36).

Analyzation methods used to study nerve regeneration may include histomorphometry analysis, immunostaining, and confocal microscopy of axons (Rajan et al., 2003) (Brenner, Michael J. et al. 2006. “Repair of Motor Nerve Gaps with Sensory Nerve Inhibits Regeneration in Rats.” Laryngoscope 116(9): 1685-92). However, functional aspects of nerve recovery are poorly correlated to the data shown in electrophysiological and histomorphometrically analysis (Dijkstra, Jeroen R., Marcel F. Meek, Peter H. Robinson, and Albert Gramsbergen. 2000. “Methods to Evaluate Functional Nerve Recovery in Adult Rats: Walking Track Analysis, Video Analysis and the Withdrawal Reflex.” Journal of Neuroscience Methods 96(2): 89-96). In addition, most methods for evaluating sensory nerve recovery are invasive and not applicable to longitudinal studies (Zeng, Li et al. “A Noninvasive Functional Evaluation Following Peripheral Nerve Repair with Electromyography in a Rat Model.”). Withdrawal response is a useful way to test for sensory nerve recovery, in which the two copper wires of a bipolar electrode are placed 3 mm apart to stimulate the foot sole in an adult rat (Dijkstra et al. 2000). The normal reaction of a rat to stimulation (0.1 mA) may be withdrawal of its foot and spread of its toes. In the first few weeks after reinnervation surgery, there was no reaction from the rats. However, some of the rats evoked a withdrawal reflex at high stimulation intensities after 3 weeks, and at 21 weeks post-op a withdrawal reflex was seen in 90% of the rats at 0.33 mA.

To validate the surgical solution and test the viability of the device with living skin, an experiment was conducted using rats to see if the implant's motion, material, and surgical techniques, may allow the skin to remain healthy over time. The aims for the rat model were to see if the flap is large enough to cover the implant but small enough to prevent necrosis, if the flap is ‘tubularized’ to imitate the human flap surrounding thumb, finger, or joint, and if the flap has sufficient motion caused by the rat's daily movements to allow for motion of implant under the skin.

The surgical procedures were designed and tested on four rat cadavers prior to starting live rat procedures. Male Lewis Rats weighing around 300 g to 480 g were used. Meloxicam and Gabapentin were administered to the rats one day before the operation. At least one hour prior to the procedure the rats were administered 72 hours sustained release of Buprenorphine, regular Buprenorphine, Meloxicam, Gabapentin, and Lactated Ringer's Solution (LRS). Rats were first anesthetized using long-duration Isoflurane. During anesthesia, a standard heating pad was used to provide heat to the rats.

To determine if this rat model may accurately represent how the skin reacts to the motion of implant under the skin, three points on the flap were tracked using tracker software from Physlets. After the rat was euthanized, three points were drawn on the ‘tubularized’ flap on its most distal, proximal, and center region.

The findings in this study provide a viable rat rotation model for a ‘tubularized’ flap with sufficient motion for prosthesis implantation. Two rats successfully underwent flap elevation surgery, and three rats successfully underwent flap rotation (model 3) surgery. No necrosis was found in all 5 rats.

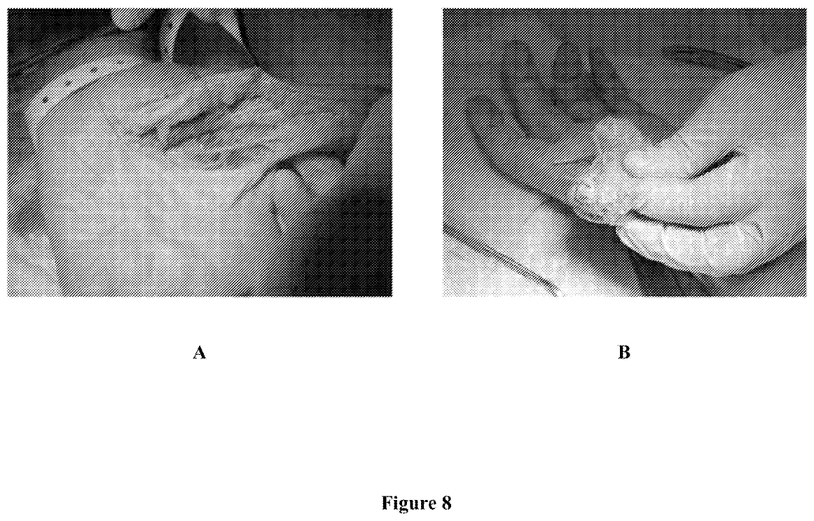

To test the implant motion in a human hand, a cadaver hand was dissected, and the prosthetic hinge thumb design was implanted into the human hand. The tendons, nerves, and muscles were identified before amputating the thumb at the proximal phalanx as shown in FIG. 8A and FIG. 8B.

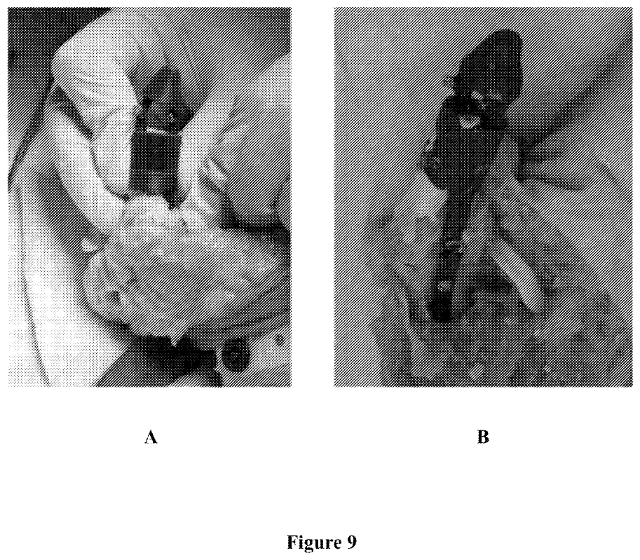

The hinge thumb prototype was 3D printed and the hinges were assembled using wires. The implant was fixated using bone cement into the medullary canal of the proximal phalanx and screwed into the volar side of the metacarpal bone as shown in FIGS. 9A and 9B.

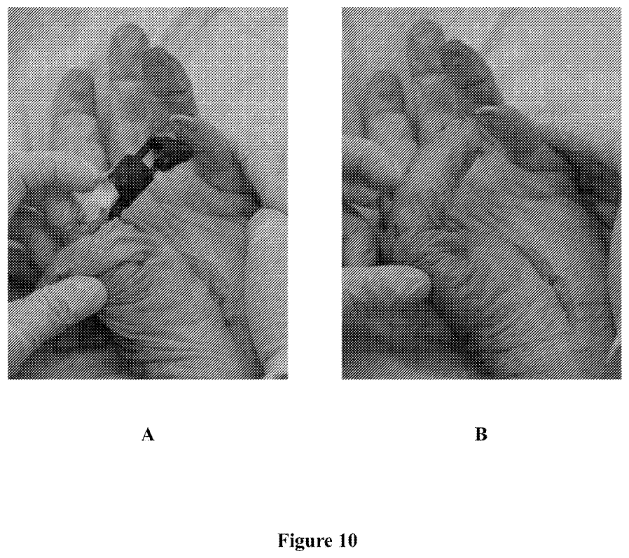

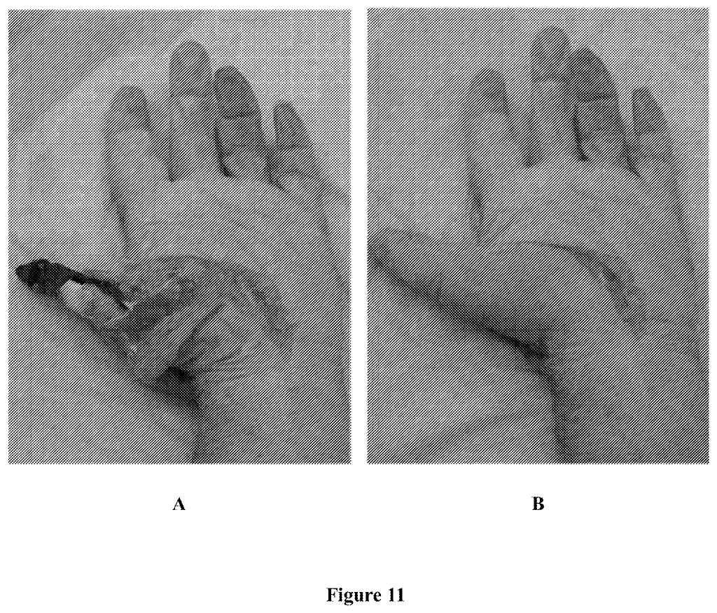

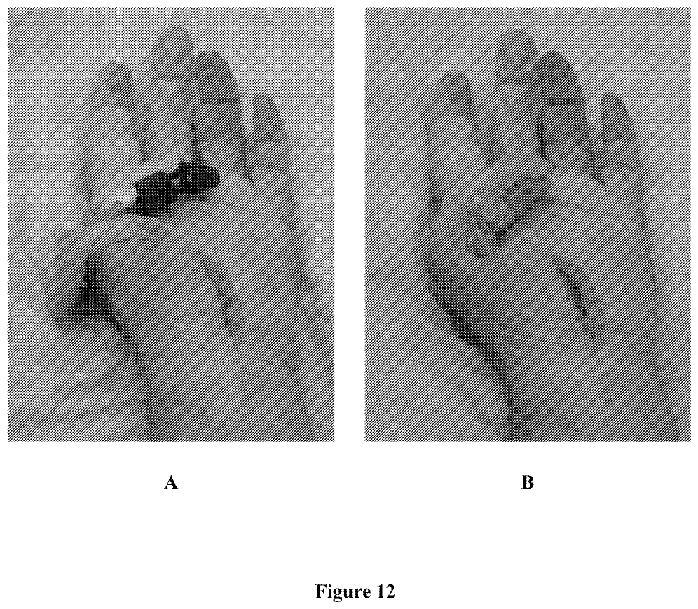

FIGS. 10A and 10B, 11A and 11B, and 12A and 12B show that this dissection successfully showed restoration of opposition motion, extension, and flexion of the thumb implant design in the human hand.

In summary, described is an implantable prosthetic design that integrates with the bone and restores joint motion. The current range of motion of the hinge design is 60°, which is more than sufficient for opposition grasp. A surgical method is also described to surround the prosthetic with sensate skin. This method restores natural sensation in amputated regions, which is a dramatic unmet need in current prosthetic devices.

This solution has the potential to extend to partial hand amputations and even full hand amputations. This can revolutionize the standard of care for people with hand amputations because it provides a solution to replace functional components associated with the natural hand anatomy and physiology. Presently, there is not a single finger prosthetic device that provides solutions in all of the following areas: direct integration of the prosthesis with the human body, cosmesis, function, sensation, and stability. This solution may create an illusion of a complete natural limb replacement.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A prosthetic device, comprising:
a first linkage unit having a central body, a distal end and a proximal end, wherein the proximal end is configured to fixate into a first bone;
a second linkage unit having a central body, a distal end and a proximal end, wherein the proximal end is configured to fixate into a second bone different from the first bone;
a distal component sized and shaped to resemble a distal phalanx;
a first hinge in the first linkage unit connecting the proximal end and the central body;
a second hinge connecting the distal end of the first linkage unit and proximal end of the distal component; and
a third hinge connecting the distal end of the second linkage unit and the proximal end of the distal component.

2. The prosthetic device of claim 1, wherein the device is implantable.

3. The prosthetic device of claims 1-2, wherein the device has a total extended length of between 3-10 centimeters.

4. The prosthetic device of claims 1-3, wherein the device has a total diameter of between 5 -25 millimeters.

5. The prosthetic device of claims 1-4, wherein the device has a length of 10-50 millimeters from the second bone to the distal component.

6. The prosthetic device of claims 1-5, wherein the second hinge is configured to revolve around the third hinge while simultaneously rotating.

7. The prosthetic device of claims 1-6, wherein the device is configured to have a larger range of motion when the second hinge is set further forward with respect to the third hinge when at rest.

8. The prosthetic device of claims 1-7, wherein the second hinge is set slightly to the side of, or straddling, the third hinge.

9. The prosthetic device of claims 1-8, wherein the first hinge is rotatable up to about 90°.

10. The prosthetic device of claims 1-9, wherein the second hinge is rotatable up to about 90°.

11. The prosthetic device of claims 1-10, further comprising a locking pin.

12. The prosthetic device of claims 1-11, wherein the device comprises metal articulating surfaces.

13. The prosthetic device of claims 1-12, wherein at least a portion of the first, second, and third hinges are comprise polyethylene.

14. The prosthetic device of claims 1-13, further comprising a coverage protecting each of the first, second, and bottom hinges.

15. The prosthetic device of claims 1-14, wherein the first bone is a metacarpal bone.

16. The prosthetic device of claims 1-15, wherein the second bone is a residual proximal phalanx.

17. The prosthetic device of claims 1-16, wherein the first linkage unit passes through at least a portion of the central body of the second linkage unit.

18. A skin-covered, linkage-driven implantable prosthesis, the prosthesis comprising:
a first linkage comprising a distal end and a proximal end, wherein the proximal end is configured to fixate into a first proximal bone and comprises a first hinge configured to rotate in parallel with a first proximal joint;
a second linkage comprising a distal end and a proximal end, wherein the proximal end is configured to fixate into a second proximal bone;
a second hinge attached to the distal end of the first linkage and configured to imitate a second proximal joint; and
a tip component configured to imitate a distal phalanx and connected to the first linkage by the second hinge and to the second linkage by a bottom hinge;
wherein motion of the prosthesis is allowed through a linkage-driven mechanism.

19. A surgical method of implanting a skin-covered, linkage-driven implantable prosthesis, the method comprising:
creating an incision of a radial forearm flap having a proximal end and a distal end;
clipping the radial artery on the proximal end of the radial forearm flap, wherein blood is supplied through the distal end of the flap where the radial artery is left intact;
elevating the radial forearm flap;
implanting the prosthesis on the elevated flap;
tubularizing the elevated flap around the prosthesis before the incision is closed;
fixating the prosthesis with the flap into a medullary canal of a first bone and a second bone; and
initiating a reinnervation procedure of the flap.

20. The method of claim 19, wherein 6 to 8 weeks pass between tubularizing the elevated flap and fixating it to the first bone and a second bone to allow for integration with the skin without the interference of motion.

21. The method of claims 19-20, wherein the reinnervation procedure includes anastomosing a lateral antebrachial cutaneous nerve in an “end to end” fashion with a digital nerve.

22. The method of claims 19-21, wherein the reinnervation procedure provides usable sensation to the prosthesis, including differentiating temperature, pressure, and pain.

23. The method of claims 19-22, wherein the prosthesis is fixated into the first bone by at least a screw.

24. The method of claims 19-23, wherein the prosthesis is fixated into the second bone using bone cement.
